(19) United States
(12) Patent Application Publication
(10) Pub. No.: US 2010/0330063 A1
(43) Pub. Date: Dec. 30, 2010

(54) STEM-LIKE CELLS, METHOD FOR DE-DIFFERENTIATING MAMMALIAN SOMATIC CELLS INTO STEM-LIKE CELLS, AND METHOD FOR DIFFERENTIATING STEM-LIKE CELLS

(75) Inventor: David E. Weinstein, Dobbs Ferry, NY (US)

Correspondence Address: GLOBAL PATENT GROUP - GLM, 1005 Warson Road, Suite 201, St. Louis, MO, 63132, US

(73) Assignee: GLIAMED, INC., New York, NY (US)

(21) Appl. No.: 12/519,499

(22) PCT Filed: Dec. 17, 2008
(86) PCT No.: PCT/US08/13843

§ 371 Date: Jun. 16, 2009

Related U.S. Application Data
(60) Provisional application No. 61/014,087, filed on Dec. 17, 2007.

(51) Int. Cl. A61K 31/401; C12N 5/02; C12Q 1/02; A61K 31/728; A61K 31/726; A61K 38/39; A61K 31/4365; A61K 38/48; A61P 19/02; A61P 19/08; A61P 25/00; A61P 25/28; A61P 1/16; A61P 27/02; A61P 35/00; A61P 17/02; A61P 1/00; A61P 35/02; A61P 9/00

(52) U.S. Cl. 424/94.64; 435/375; 435/29; 435/325; 514/423; 514/54; 514/17.2; 514/301

(57) ABSTRACT

A new use is provided for small molecule inhibitors of Oct4 and Sox 2 as a cellular reprogramming agent and a method of reprogramming adult mammalian somatic cells into stem-like cells is provided, using small molecule inhibitors of Oct4 and Sox 2 without the need of any material derived from embryos or fetuses, and without the need of potentially harmful transfecting vectors. Stem-like cells created by the present invention can be induced to differentiate into terminally differentiated adult somatic cells, such as, for example, neuronal cells.

This application claims the benefit of priority of U.S. provisional application No. 61/014,087, filed Dec. 17, 2007, the disclosure of which is hereby incorporated by reference as if written herein in its entirety.

Disclosed herein are new uses for immunophilin ligands including GM1485 as reprogramming agents and methods of reprogramming adult mammalian fibroblasts into stem-like cells using immunophilin ligands, without the need for any material derived from embryos or fetuses and without the need for potentially harmful transfecting vectors, viruses or viral elements. Stem-like cells created by the methods disclosed herein can be induced to reprogrammed into terminally differentiated adult somatic cells, such as, for example, neuronal cells. Regenerative medicine's potential for alleviating human suffering turns almost entirely on directing the growth of stem cells into terminally differentiated specialized somatic tissues. Consequently, stem cells may be regarded as the raw material of regenerative medicine. Because the harvesting of stem cells from human embryos has been fraught with ethical controversy, an ethically acceptable alternative to harvesting stem cells for research and medical treatment is highly desirable, particularly if the alternative can provide human stem cells in abundance.

Since the isolation of embryonic stem cells, it has been discovered that mammalian adult cells of non-embryonic origin also have the potential of differentiating into more specialized types of cells. As to these, it has been hypothesized that embryonic stem cells are deposited into certain tissue compartments during gastrulation, where they remain throughout life in a potentially reversible state of dormancy.

Cells with the potential for differentiating into different cells types have also been produced by the biochemical or genetic manipulation of adult somatic cells. For example, U.S. patent application Ser. No. 11/055,454 by Dominko and Page, entitled, De-differentiation and re-differentiation of somatic cells and production of cells for cell therapies, filed on Feb. 9, 2005, and published on May 26, 2006 as U.S. Pat. Pub. No. 20060110830, discloses a method for reprogramming somatic cells by culturing the cells by introducing components of the cytoplasm of pluripotent cells into the somatic cells, and allowing the cells to reprogram. The pluripotent cells may be blastomeres, inner cell mass cells, embryonic stem cells, embryonic germ cells, embryos, embryoid body cells, morula-derived cells, and multipotent partially differentiated embryonic stem cells taken in the embryonic development process. This method suffers from the drawback that the reprogramming-inducing agents must still be harvested from human embryos.

Several weeks before the filing of the instant application, the press reported that Shinya Yamanaka and James A. Thomson had developed a method for genetically reprogramming human skin cells to reprogram into pluripotent stem cells. However, this technique suffers from the drawback that the genetic reprogramming entails the use of a retrovirus having a carcinogenic potential (Gina Kolata, Scientists Bypass Need for Embryo to Get Stem Cells, New York Times, Nov. 21, 2007.

Accordingly, regenerative medicine is still in profound need of a utilitarian method of providing stem cells, particularly from a patient's own somatic cells, without the risk of carcinogenesis and without engendering ethical conflict. Provided herein are methods which meet this need. These methods include a method that creates stem-like cells from adult mammalian fibroblasts by culturing the fibroblasts in vitro in the presence of an immunophilin ligand. Also provided is a use for immunophilin ligands as reprogramming agents.

### BRIEF DESCRIPTION OF THE DRAWINGS

FIG. 1 shows a 2 photomicrographs. The left photomicrograph shows human fibroblasts grown with GM1485. The right photomicrograph shows murine fibroblasts grown with a vehicle. Cells grown under these conditions were stained with an αOct4 antibody. Only the cells treated with GM1485 express Oct4.

FIG. 2 shows four photomicrographs arrayed in a matrix of two rows and two columns. The images in the upper row are of cells that had been treated with GM1485 and subsequently cultured under neural inducing conditions. The photomicrographs in the bottom row had been treated identically with the exception that they were never exposed to GM1485. The cells in the photomicrographs in the left column were stained with an antiserum that recognizes the astrocyte-specific protein glial fibrillary acidic protein (GFAP). The cells in the photomicrographs in the right column were stained with TuJ1, a mouse monoclonal antibody that recognizes neuron-specific βIII tubulin. The photomicrograph insets in the bottom row of photomicrographs are the Hoescht stained nuclei of the cells in the photomicrographs in which they are set. The cells in these photomicrographs in the lower row were not treated with GM1485, and thus were not reprogrammed along a neural pathway. As a result, these cells express neither GFAP nor TuJ1.

FIG. 3 shows two photomicrographs. The left photomicrograph shows murine fibroblasts grown with GM1485. The right photomicrograph shows murine fibroblasts grown with vehicle. Cells grown under these conditions were stained with an αOct4 antibody. Only the cells treated with GM1485 express Oct4.

FIG. 4. The left panel in FIG. 4 shows an H&E stained section of a vehicle treated heart, and the right panel shows an H&E stained section of a GM1485 treated heart.

FIG. 5 is a 2×2 matrix of photomicrographs of cardiac tissue 30 days after infarction and treatment with either vehicle in the top two slides or GM1485 in the bottom two slides. The left upper and lower sections were stained with an αOct4 antiserum and the right upper and lower sections were stained with αSox2.

Provided herein is a method for reprogramming a mammalian somatic cell into a stem-like cell, comprising introducing a small molecule which induces the expression of Oct4.

Also provided herein is a method for reprogramming a mammalian somatic cell into a stem-like cell, comprising introducing a small molecule which induces the expression of Sox2

In certain embodiments, the small molecule is an immunophilin ligand.

In certain embodiments, said method is practiced in vitro and additionally comprises the step of culturing the mammalian somatic cell.

In certain embodiments, the method further comprises, after the step of introducing a small molecule which induces the expression of either Oct4 or Sox2, culturing the mammalian somatic cell under conditions suitable for maintaining pluripotent stem cells in an undifferentiated state.

In certain embodiments, the method further comprises, after the step of introducing a small molecule which induces the expression of either Oct4 or Sox2, culturing the mammalian somatic cell under conditions that induce or direct partial or complete differentiation to a particular cell type.

In certain embodiments, the method further comprises, after the step of introducing a small molecule which induces the expression of either Oct4 or Sox2, culturing the mammalian somatic cell in a serum-free neuro-differentiation medium.

In certain embodiments, the method further comprises, after the step of culturing the mammalian somatic cell in a serum-free neuro-differentiation medium, culturing the mammalian somatic cell in the neuro-differentiation medium without FGF2.

In certain embodiments, the method further comprises, after the step of culturing the mammalian somatic cell in a serum-free neuro-differentiation medium, culturing the mammalian somatic cell in the neuro-differentiation medium with FGF2.

In certain embodiments, the method further comprises, after the step of culturing the mammalian somatic cell in the neuro-differentiation medium without FGF2, culturing the mammalian somatic cell the neuro-differentiation medium without FGF2 and augmented with rhEGF.

In certain embodiments, the method further comprises, after the step of culturing the mammalian somatic cell in the neuro-differentiation medium without FGF2 and augmented with rhEGF, culturing the mammalian somatic cell the neuro-differentiation medium without FGF2 and augmented with rhEGF and an immunophilin ligand.

In certain embodiments, the mammalian somatic cell is selected from the group consisting of fibroblasts, B cells, T cells, dendritic cells, keratinocytes, adipose cells, epithelial cells, epidermal cells, chondrocytes, neural cells, cardiac cells, esophageal cells, muscle cells, melanocytes, hematopoietic cells, macrophages, monocytes, and mononuclear cells.

In certain embodiments, the mammalian somatic cell is a fibroblast.

In certain embodiments, the mammalian somatic cell is a dermal fibroblast.

In certain embodiments, the immunophilin ligands have structural Formula I:

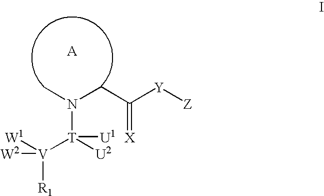

or a salt, ester, or amide thereof, wherein:

A is a saturated or unsaturated five- to seven-membered monocyclic heterocycloalkyl containing, in addition to the nitrogen atom shown, one to three heteroatoms selected from the group consisting of nitrogen, oxygen, and sulfur, and may be optionally substituted;

U¹ and U² are each independently selected from the group consisting of singly or doubly bonded 0, singly or doubly bonded S, C₁-C₆ straight or branched chain alkyl, C₃-C₆ cycloalkyl, C₃-C₆ heterocycloalkyl, phenyl, benzyl, and C₅-C₆ heteroaryl, —(C₁-C₆ straight alkyl)-(C₃-C₆ cycloalkyl), —(C₁-C₆ straight alkyl)-(C₃-C₆ heterocycloalkyl), —(C₁-C₆ straight alkyl)-phenyl, —(C₁-C₆ straight alkyl)-benzyl, and —(C₁-C₆ straight alkyl)-(C₅-C₆ heteroaryl), any of which may be optionally substituted with one to three substituents selected from the group consisting of halogen, trifluoromethyl, C₁-C₄ straight or branched chain alkyl, C₁-C₄ straight or branched chain alkoxy, C₁-C₄ straight or branched chain haloalkoxy, and cyano; or either or both of U¹ and U² may be absent;

W¹ and W² are each independently selected from the group consisting of hydrogen, singly or doubly bonded O, singly or doubly bonded S, C₁-C₆ straight or branched chain alkyl, C₃-C₆ cycloalkyl, C₃-C₆ heterocycloalkyl, phenyl, benzyl, and C₅-C₆ heteroaryl, —(C₁-C₆ straight alkyl)-(C₃-C₆ cycloalkyl), —(C₁-C₆ straight alkyl)-(C₃-C₆ heterocycloalkyl), —(C₁-C₆ straight alkyl)-phenyl, —(C₁-C₆ straight alkyl)-benzyl, and —(C₁-C₆ straight alkyl)-(C₅-C₆ heteroaryl), any of which may be optionally substituted with one to three substituents selected from the group consisting of halogen, trifluoromethyl, C₁-C₄ straight or branched chain alkyl, C₁-C₄ straight or branched chain alkoxy, C₁-C₄ straight or branched chain haloalkoxy, and cyano; or either or both of W¹ and W² may be may be absent;

T is selected from the group consisting of C, S, and N;

V is selected from the group consisting of C, N, and a bond;

X is selected from the group consisting of O, and S; or X may be two hydrogens bonded to the parent carbon;

Y is selected from the group consisting of O, C(O), and S;

Z is selected from the group consisting of hydrogen, C₃-C₉ straight or branched chain alkyl, C₃-C₆ cycloalkyl, C₅-C₇ cycloalkenyl, C₃-C₆ heterocycloalkyl, phenyl, benzyl, and C₅-C₆ heteroaryl, —(C₁-C₆ straight alkyl)-(C₃-C₆ cycloalkyl), —(C₁-C₆ straight alkyl)-(C₃-C₆ heterocycloalkyl), —(C₁-C₆ straight alkyl)-phenyl, —(C₁-C₆ straight alkyl)-benzyl, and —(C₁-C₆ straight alkyl)-(C₅-C₆ heteroaryl), any of which may be optionally substituted with one to three substituents selected from the group consisting of halogen, trifluoromethyl, C₁-C₄ straight or branched chain alkyl, C₁-C₄ straight or branched chain alkoxy, C₁-C₄ straight or branched chain haloalkoxy, and cyano; and

R¹ is selected from the group consisting of C₃-C₉ straight or branched chain alkyl or alkenyl, C₃-C₆ cycloalkyl, C₅-C₇ cycloalkenyl, C₃-C₆ heterocycloalkyl, phenyl, benzyl, and C₅-C₆ heteroaryl, —(C₁-C₆ straight alkyl)-(C₃-C₆ cycloalkyl), —(C₁-C₆ straight alkyl)-(C₃-C₆ heterocycloalkyl), —(C₁-C₆ straight alkyl)-phenyl, —(C₁-C₆ straight alkyl)-benzyl, and —(C₁-C₆ straight alkyl)-(C₅-C₆ heteroaryl), any of which may be optionally substituted with one to three substituents selected from the group consisting of halogen, trifluoromethyl, C₁-C₄ straight or branched chain alkyl, C₁-C₄ straight or branched chain alkoxy, C₁-C₄ straight or branched chain haloalkoxy, and cyano.

In further embodiments, the immunophilin ligands have any one of structural Formulas II-VI:

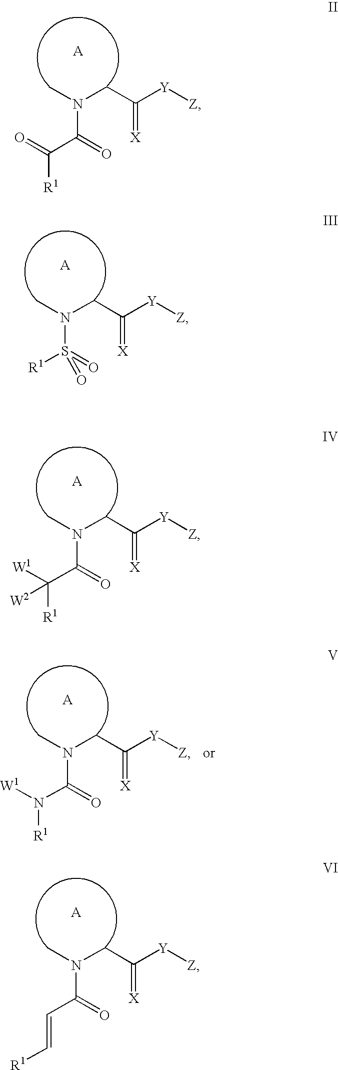

or a salt thereof, wherein:

A is selected from the group consisting of pyrrole, pyrroline, pyrrolidine, pyrazole, pyrazoline, pyrazolidine, imidazole, imidazoline, imidazolidine, oxazole oxazoline, oxazolidine, isoxazole, isoxazoline, isoxazolidine, thiazole, thiazoline, thiazolidine, isothiazole, isothiazoline, isothiazolidine, triazole, oxathiazole, thiadiazole, dithiazole, piperidine, piperazine, morpholine, thiomorpholine, pyridine, piperazine, pyridazine, pyrimidine, pyrazine, azepine, and diazepine, and the saturated and unsaturated equivalents thereof; and

all other groups are as previously defined.

In further embodiments, the immunophilin ligands have any one of structural Formulas II-XI:

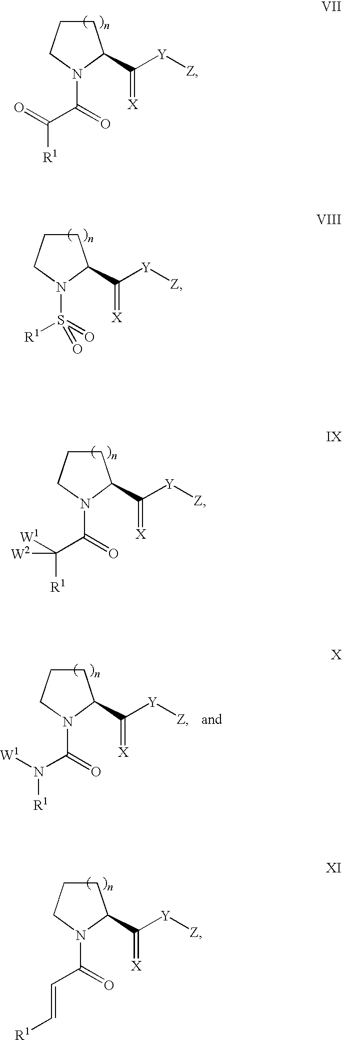

or a salt thereof, wherein:

n is an integer from 1 to 3;

and all other groups are as previously defined.

In further embodiments, n is 1.

In further embodiments, Y is O.

In yet further embodiments, X is O.

In further embodiments, Y is S.

In yet further embodiments, X is O.

In yet further embodiments, X is S.

In other embodiments, n is 2.

In further embodiments, Y is O.

In yet further embodiments, X is O.

In further embodiments, Y is S.

In yet further embodiments, X is O.

In yet further embodiments, X is S.

In yet further embodiments, the immunophilin ligands have any one of structural Formulas XII-XVI:

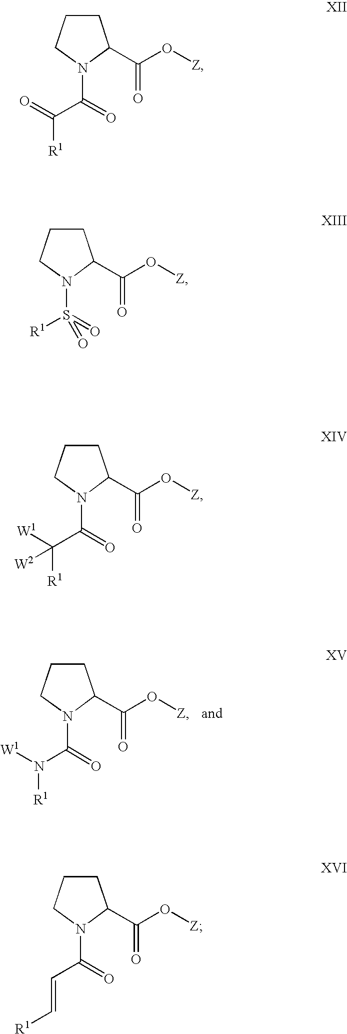

or a salt, ester, or amide thereof, wherein:

R¹ represents a C₃-C₉ straight or branched chain alkyl or alkenyl group optionally substituted with C₃-C₇ cycloalkyl, C₅-C₇ cycloalkenyl, or phenyl, any of which may be optionally substituted with C₁-C₄ alkyl, C₁-C₄ alkenyl, C₁-C₄ alkoxy, C₁-C₄ haloalkyl, C₁-C₄ haloalkoxy, perfluoromethyl, perfluoromethoxy, halogen, cyano, or hydroxyl; and

Z represents hydrogen or a C₃-C₆ straight or branched chain alkyl or alkenyl group optionally substituted with C₃-C₆ cycloalkyl, or phenyl, any of which may be optionally substituted with C₁-C₄ alkyl, C₁-C₄ alkoxy, C₁-C₄ haloalkyl, C₁-C₄ haloalkoxy, perfluoromethyl, perfluoromethoxy, halogen, cyano, or hydroxyl.

In yet further embodiments, the immunophilin ligands have structural Formula XII:

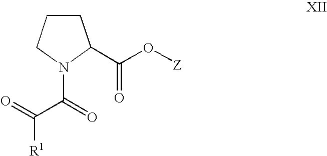

or a salt or ester, thereof, wherein:

R¹ represents a C₃-C₆ straight or branched chain alkyl or alkenyl group optionally substituted with C₃-C₇ cycloalkyl, C₅-C₇ cycloalkenyl, or phenyl, any of which may be optionally substituted with C₁-C₄ alkyl, C₁-C₄ alkenyl, C₁-C₄ alkoxy, C₁-C₄ haloalkyl, C₁-C₄ haloalkoxy, perfluoromethyl, perfluoromethoxy, halogen, cyano, or hydroxyl; and

Z represents hydrogen.

In further embodiments, R¹ is 1,1-dimethylpropyl and Z represents hydrogen, i.e., the immunophilin ligand is (2S)-1-(1,2-dioxo-3,3-dimethylpentyl)-2-pyrrolidine carboxylic acid (hereinafter “GM1485”).

In further embodiments, the compound is selected from the group consisting of:
(2S)-1-(3,3-dimethyl-1,2-dioxopentyl)-2-pyrrolidine carboxylic acid,
(2S)-1-(1,2-dioxo-2-cyclohexyl)ethyl-2-pyrrolidinecarboxylic acid,
(2S)-1-(1,2-dioxo-4-cyclohexyl)butyl-2-pyrrolidinecarboxylic acid,
(2S)-1-(3,3-dimethyl-1,2-dioxo-4-hydroxybutyl)-2-pyrrolidine carboxylic acid,
(2S)-1-(3,3-dimethyl-1,2-dioxopentyl)-2-pyrrolidine carboxamide,
1-[1-(3,3-dimethyl-1,2-dioxopentyl)-L-proline]-L-phenylalanine
1-[1-(3,3-dimethyl-1,2-dioxopentyl)-L-proline]-L-leucine,
1-[1-(3,3-dimethyl-1,2-dioxopentyl)-L-proline]-L-phenylglycine,
1-[1-(3,3-dimethyl-1,2-dioxopentyl)-L-proline]-L-phenylalanine, and
1-[1-(3,3-dimethyl-1,2-dioxopentyl)-L-proline]-L-isoleucine.

In certain embodiments, the mammal is a human.

Further provided is a method for reprogramming a mammalian somatic cell to become a cell of neural lineage, comprising:
(a) culturing a mammalian somatic cell that is not of neural lineage,
(b) introducing a small molecule which induces the expression of Oct4,
(c) culturing the cell in a serum-free neuro-differentiation medium,
(d) replacing the serum-free neuro-differentiation medium with serum-free neuro-differentiation medium with FGF2,
(e) replacing the serum-free neuro-differentiation medium without FGF2 with serum-free neuro-differentiation medium with FGF2 and augmented with rhEGF,
(f) replacing the serum-free neuro-differentiation medium without FGF2 with serum-free neuro-differentiation medium with FGF2 and augmented with rhEGF,
(g) replacing the serum-free neuro-differentiation medium without FGF2 and augmented with rhEGF with serum-free neuro-differentiation medium without FGF2 and augmented with rhEGF and an immunophilin ligand.

In certain embodiments, said small molecule which induces the expression of either Oct4 or Sox2 is an immunophilin ligand.

In certain embodiments, the method further comprises assaying to detect a marker of cells of neural lineage.

Further provided is a composition of cells of neural lineage prepared by the methods disclosed herein.

Yet further provided is a composition comprising stem-like cells prepared by the methods disclosed herein.

Also provided is a method of treatment of a cellular degenerative disorder by the reprogramming of mammalian somatic cell, comprising the administration of a therapeutically effective amount of a small molecule which induces the expression of either Oct4 or Sox2 to a patient in need thereof.

In certain embodiments, said small molecule which induces the expression of either Oct4 or Sox2 is an immunophilin ligand.

Also provided is a method of treatment, by the reprogramming of mammalian somatic cell, of a disease selected from the group consisting of osteoarthritis, bone fractures, non-union bone fractures, articular trauma, acute coronary syndrome, occlusive stroke, spinal cord injury, traumatic brain injury, peripheral nerve trauma, non-autoimmune demyelinating diseases, acute amyotrophic sclerosis, Huntington's Disease, Alzheimer's Disease, Guillain-Barrè Syndrome, transverse myelitis, hepatic cirrhosis, hepatic fibrosis, macular degeneration, retinal trauma, diabetic retinopathy, actinic keratosis, basal cell carcinoma, keloid scarring, enhanced scar reduction, burns, diabetic ulcers, stasis ulcers, venous ulcers, peptic ulcer disease, duodenal ulcer disease, esophageal lesions, irritable bowel syndrome, periodontal disease, dental implants, acute myelocytic leukemia, acute promylocytic leukemia, breast cancer, cervical cancer, lymphoid cancers and other diseases in which tissue regeneration is a component of healing comprising the administration of a therapeutically effective amount of a small molecule which induces the expression of either Oct4 or Sox2 to a patient in need thereof.

In certain embodiments, said small molecule which induces the expression of Oct4 is an immunophilin ligand.

Further provided is a method of treatment of a diseases by the reprogramming of mammalian somatic cell, comprising the administration of:
a therapeutically effective amount of a small molecule which induces the expression of either Oct4 or Sox2; and
another therapeutic agent.

In certain embodiments, said other agent is selected from the group consisting of topical or injectable lidocaine, a topical antibiotic, hyaluronan, a long-chain polymer containing repeating disaccharide units of Na-glucuronate-N-acetylglucosamine, with or without chondroitin sulfate, a hydrogel, a nonsteroidal anti-inflammatory drug, collagens or synthetic fillers, topical or oral retinoids, sodium bicarbonate, pressor agents, Plavix®, a tissue plasminogen activator, streptokinase, and a drug for the treatment of an acute coronary syndromes.

Also provided is a method for achieving an effect in a patient by the reprogramming of mammalian somatic cell, comprising the administration of a therapeutically effective amount of a small molecule which induces the expression of either Oct4 or Sox2 to a patient, wherein the effect is selected from the group consisting of enhanced regeneration of donor liver in living-donor liver transplantation, enhanced regeneration of recipient liver in living-donor liver transplantation renal degenerative diseases, enhanced regeneration of skin-graft donor sites, dermal regeneration following surgical or traumatic wounds.

In certain embodiments, said small molecule which induces the expression of either Oct4 or Sox2 is an immunophilin ligand.

In certain embodiments, said immunophilin ligand is GM1485.

GM1485 is disclosed in U.S. Pat. No. 5,614,457. GM1485 is claimed as a molecular composition of matter and a pharmaceutically acceptable salt or hydrate in U.S. Pat. No. 7,282,510, whose content is incorporated herein by reference. GM1485 was known to demonstrate several biochemical and pharmacologic activities, such as, for example, neurotrophic activity. However, its reprogramming activity was heretofore unknown.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl may comprise from, for example, 1 to 6 carbon atoms or 3 to 9 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The terms “benzo” and “benz,” as used herein, alone or in combination, refer to the divalent radical C₆H₄═ derived from benzene. Examples include benzothiophene and benzimidazole.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. Unless otherwise specified, the term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur Heteroaryl groups are a subset of heterocycles. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydroxy,” as used herein, alone or in combination, refers to —OH.

The term “lower,” as used herein, alone or in a combination, where not otherwise specifically defined, means containing from 1 to and including 6 carbon atoms.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “sulfonyl,” as used herein, alone or in combination, refers to —S(O)₂—.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, unless otherwise so designated, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “regenerative immunophilin ligand” is used herein to refer to a compound that binds to the intracellular protein FKBP52, and exerts a proregenerative activity as measured by its affects on enhancing wound healing (see U.S. Pat. No. 7,189,746) or by enhancing nerve regeneration in vivo. The binding affinity of the ligand to FKBP52, K_{d}, can be measured in vitro by one knowledgeable in the art, and the biologic activity can be measured as described in U.S. Pat. No. 7,189,746 and U.S. Pat. No. 6,569,423, or by one knowledgeable in the art.

The term “reprogram” means to alter the cellular identity or fate.

The term “cell of neural lineage” refers to neurons and glial cells, including astrocytes, oligodendrocytes, ependymal cells, radial glia, Schwann cells, and satellite cells.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is rendered more active in vivo, after administration to a patient. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally skin, nasal or the buccal cavities and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the stratum corneum to the site of inflammation in the skin such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 6 g/day. Tablets or other forms of presentation provided in discrete units may conveniently contain an amount of one or more compounds which is effective at such dosage or as a multiple of the same, for instance, units containing 5 mg to 1000 mg, usually around 10 mg to 500 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant practitioner. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

topical anesthetics such as lidocaine for topical treatment; commercial available topical excipients, such as Amantle®; topical antibiotic creams and ointments such as mupirocin (Bactroban®); intra-articular administration of artificial synovial fluid or derivatives of hyaluronan (sodium hyaluronate), a long-chain polymer containing repeating disaccharide units of Na-glucuronate-N-acetylglucosamine, with or without chondroitin sulfate, such as Synvisc® or Provisc®; intra-ocular administration with a hydrogel such as Healon® or application to the cornea with hydrogel and nonsteroidal anti-inflammatory drug such a diclofen (Voltaren®), or combined with hydrogel contact lenses; tissue fillers such as collagens or synthetic fillers such as Restylane®; with topical or oral retinoids such as Accutane® or Targretin® (bexarotene); injectable lidocaine, sodium bicarbonate, various pressor agents, Plavix®, tissue plasminogen activators, streptokinase and other compounds used in the setting of acute coronary syndromes.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Specific diseases to be treated by the compounds, compositions, and methods disclosed herein include those of the circulatory, digestive, endocrine, integument, muscular, nervous, reproductive, respiratory, skeletal and urinary systems. These diseases may be congenital in nature or relate to later onset. In addition, the compounds, compositions, and methods disclosed herein may be used to treat injury to those same organ systems.

Thus, in another aspect, certain embodiments provide methods for the promotion of dermal regeneration (wound healing) in a human or animal subject in need of such treatment comprising administering to said subject an amount of a compound disclosed herein effective to induce and enhance regeneration in the subject, in combination with at least one additional agent for the treatment of said disorder that is known in the art. In a related aspect, certain embodiments provide therapeutic compositions comprising at least one compound disclosed herein in combination with one or more additional agents for the treatment of acute surgical or traumatic wounds.

Specific diseases to be treated by the compounds, compositions, and methods disclosed herein include osteoarthritis, bone fractures, non-union bone fractures, articular trauma, acute coronary syndrome, occlusive stroke, spinal cord injury, traumatic brain injury, peripheral nerve trauma, non-autoimmune demyelinating diseases, acute amyotrophic sclerosis, Huntington's Disease, Alzheimer's Disease, Guillain-Barrè Syndrome, transverse myelitis, hepatic cirrhosis, hepatic fibrosis, enhanced regeneration of donor liver in living-donor liver transplantation, enhanced regeneration of recipient liver in living-donor liver transplantation renal degenerative diseases, macular degeneration, retinal trauma, diabetic retinopathy, dermal regeneration following surgical or traumatic wounds, actinic keratosis, basal cell carcinoma, keloid scarring, enhanced scar reduction, burns, enhanced regeneration of skin-graft donor sites, diabetic ulcers, stasis ulcers, venous ulcers, peptic ulcer disease, duodenal ulcer disease, esophageal lesions, irritable bowel syndrome, periodontal disease, dental implants, acute myelocytic leukemia, acute promylocytic leukemia, breast cancer, cervical cancer, lymphoid cancers and other diseases in which tissue regeneration is a component of healing.

Additionally, the compounds, compositions, and methods disclosed herein can be used in situations where heart muscle has been damaged by injury or lack of oxygenation due to blockage of supplying blood vessels. Replacement or supplement of the damaged tissue is within the scope of the invention. In another aspect, hematopoietic cells of the circulatory system may be too low in number to support normal function. Such instants may occur due to treatment of a patient with chemotherapy, immunosuppressive drugs or irradiation. Generation of appropriate cell types by use of the invention could supplement cardiovascular function. In a similar aspect, injury or disease may necessitate the re-establishment of circulation to the affected tissue. In one aspect of the invention, angiogenesis may be augmented using compounds, compositions, and methods disclosed herein. Disease and injury of the digestive system may also benefit from compounds, compositions, and methods disclosed herein. For instance, the liver could be damaged by viral infection, drug or alcohol consumption, or by physical trauma. Augmentation of liver cells using the compounds, compositions, and methods disclosed herein could help restore proper function. In addition, type 1 diabetes is caused by an autoimmune attack on the islet cells of the pancreas. Use of compounds, compositions, and methods disclosed herein to generate islet cell function could help supply needed insulin in cases of diabetes. Other autoimmune diseases where tissue damage or destruction is present may benefit from the compounds, compositions, and methods disclosed herein. As noted, treatment for some diseases may include additional drugs or treatments to control the autoimmune reaction. Some commonly identified autoimmune diseases include but are not limited to type 1 diabetes, rheumatoid arthritis, multiple sclerosis, Addison's disease, Graves' disease, celiac disease, Hashimoto's disease, lupus erythematosus, myasthenia gravis, pemphigus vulgaris, Sjogren's syndrome and vitiligo. Disease or injury to the endocrine system could benefit from the compounds, compositions, and methods disclosed herein. In one aspect, individuals with pituitary gland or thyroid gland insufficiencies could be treated. Disease and injuries of the integument system such as burns and wound healing could benefit from the compounds, compositions, and methods disclosed herein as could conditions were hair regrowth was required. Disease and injury of the muscular system such as the various muscular dystrophies could benefit compounds, compositions, and methods disclosed herein. Conditions such as Becker's, Duchenne or limb-girdle dystrophies, as well as other myopathies, are considered targets. Severe muscle injury could also benefit. The nervous system, both central and peripheral, could benefit from compounds, compositions, and methods disclosed herein. For instance, spinal cord injuries or nerve injuries of the extremities could be treated using compounds, compositions, and methods of the invention. In one aspect, degenerative diseases such as Parkinson's disease or Alzheimer's disease could be treated. In another aspect, nervous system diseases resulting from infection could be treated. In yet another aspect, diseases resulting from genetic disorders, such as Huntington disease, could be treated. In still another aspect, injury to the brain could be treated. Such injury could result from physical injury or from lack of oxygenation such as under stroke conditions. Diseases of the reproductive system could benefit from compounds, compositions, and methods disclosed herein. For instance, individuals with reproductive hormone insufficiencies could be treated. Disease of the respiratory system could be targets for treatment using the compounds, compositions, and methods disclosed herein. In one aspect, lung injury due to disease such as cystic fibrosis or lung injury due to smoking could be treated (chronic obstructive pulmonary disease). Lung injury could include the trachea as well as the bronchi tissue. Tissues of the skeletal system could be subject to treatment with the compounds, compositions, and methods disclosed herein. As mentioned destruction of the synovium by disease conditions such as rheumatoid arthritis are indications for implementation of the invention. Bone healing, joint remodeling and grafting procedures are all conditions that could benefit from the invention. Procedures requiring ligament reattachment or repair could also benefit from the invention. In addition, disease and injury of the urinary system could benefit from the compounds, compositions, and methods disclosed herein. For instance, kidney diseases are prevalent and rescue of kidney function using aspects of the invention could lead to increases quality and length of life. Kidney diseases can be genetic in nature such as polycystic kidney disease or be the result of infection or injury. As mentioned, kidney damage is often associated with Lupus, an autoimmune disease.

In practice, the compounds, compositions, and methods disclosed herein include the use as part of a medical device. The medical device can be designed for implantation into the body or can be used to function outside of the body as an approach to treating the diseases and injuries described above. When used outside of the body, the device containing compounds, compositions, and methods disclosed herein would be attached to the appropriate site on the body in order to carry out the prescribed function. For instance, it is envisioned that kidney cells derived from the invention could be enclosed in a medical device that would attach to a patients circulatory system to help purify the blood of toxins. Such device need not be implanted into the patient but could reside on the outside of the body. In other instances, for example in bone healing, the compounds, compositions, and methods disclosed herein, could be part of a medical device that is inserted into the bone tissue to augment healing. For applications that require contact with the skin, the compounds, compositions, and methods disclosed herein may be contained in a patch that adheres to the skin.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### General Synthetic Methods for Preparing Compounds

The following schemes can be used to practice the present invention.

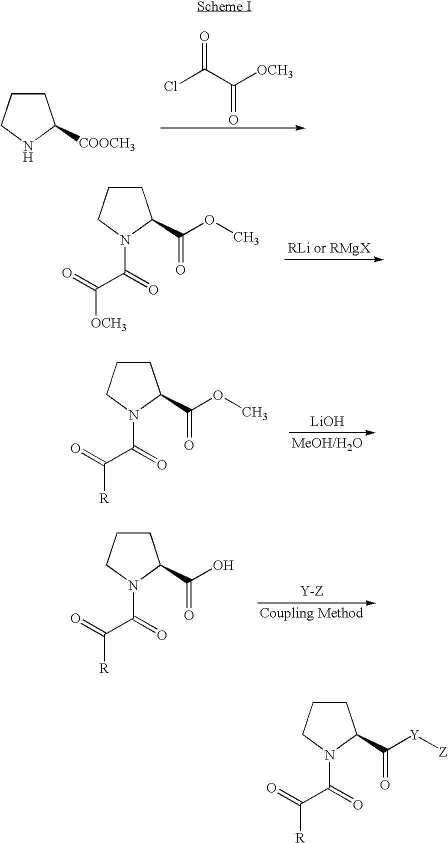

Example disclosed herein can be synthesized using the following general synthetic procedure set forth in Scheme I.

The invention is further illustrated by the following examples.

### EXAMPLE 1

### Preparation of GM1485

### (2S)-1-(3,3-dimethyl-1,2-dioxopentyl)-2-pyrrolidinecarboxylic acid

### Step 1: synthesis of methyl (2S)-1-(1,2-dioxo-2-methoxyethyl)-2-pyrrolidinecarboxylate

A solution of L-proline methyl ester hydrochloride (3.08 g; 18.60 mmol) in dry methylene chloride was cooled to 0° C. and treated with triethylamine (3.92 g; 38.74 mmol; 2.1 eq.). After stifling the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hr. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): d 1.93 (dm, 2H), 2.17 (m, 2H), 3.62 (m, 2H), 3.71 (s, 3H), 3.79, 3.84 (s, 3H total), 4.86 (dd, 1H, J=8.4, 3.3).

### Step 2: Synthesis of methyl (2S)-1-(1,2-dioxo-3,3-dimethylpentyl)-2-pyrrolidinecarboxylate

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil. ¹H NMR (CDCl₃): d 0.88 (t, 3H), 1.22, 1.26 (s, 3H each), 1.75 (dm, 2H), 1.87-2.10 (m, 3H), 25 2.23 (m, 1H), 3.54 (m, 2H), 3.76 (s, 3H), 4.52 (dm, 1H, J=8.4, 3.4).

### Step 3: synthesis of (2S)-1-(1,2-dioxo-3,3-dimethylpentyl)-2-pyrrolidinecarboxylic acid

A mixture of methyl (2S)-1-(1,2-dioxo-3/3-dimethylpentyl)-2-pyrrolidinecarboxylate (2.10 g; 8.23 mmol), 1 M LiOH (15 mL), and methanol (50 mL) was stirred at 0° C. for 30 min and at room temperature overnight. The mixture was acidified to pH 1 with 1 M HCl, diluted with water, and extracted into 100 mL of methylene chloride. The organic extract was washed with brine and concentrated to deliver 1.73 g (87%) of snow white solid which did not require further purification. 10 1H NMR (CDCl₃): d 0.87 (t, 3H); 1.22, 1.25 (s, 3H each); 1.77 (dm, 2H); 2.02 (m, 2H); 2.17 (m, 1H); 2.25 (m l 1H); 3.53 (dd, 2H, J=10.4 1 7.3); 4.55 (dd, 1H, J=8.6 1 4.1).

### EXAMPLE 2

### Preparation of an exemplary ester of GM1485 3-phenyl-1-propyl (2S)-1-(3,3-dimethyl-1,2-dioxopentyl)-2-pyrrolidinecarboxylate

(Example 1). A mixture of (2S)-1-(1,2-dioxo-3,3-dimethylpentyl)-2-pyrrolidinecarboxylic acid (600 mg; 2.49 mmol), 3-phenyl-1-propanol (508 mg; 3.73 mmol), dicyclohexylcarbodiimide (822 mg; 3.98 mmol), camphorsulphonic acid (190 mg; 0.8 mmol) and 4-dimethylaminopyridine (100 mg; 0.8 mmol) in methylene chloride (20 mL) was stirred overnight under a nitrogen atmosphere. The reaction mixture was filtered through Celite to remove solids and concentrated in vacuo, and 25 the crude material was purified on a flash column (25% ethyl acetate in hexane) to obtain 720 mg (80%) of Example 1 as a colorless oil. ¹H NMR (CDCl₃): d 0.84 (t, 3H); 1.19 (s, 3H); 1.23 (s, 3H); 1.70 (dm, 2H); 1.98 (m, 5H); 2.22 (m, 1H); 2.64 (m, 2H); 3.47 (m, 2H); 4.14 (m, 2H); 4.51 (d, 1H); 7.16 (m, 3H); 7.26 (m, 2H).

### EXAMPLE 3

### Esters of GM1485, Generally

Esters of GM1485 may be prepared as disclosed above, yielding compounds as disclosed in columns 19-24 of U.S. Pat. No. 7,282,510.

### EXAMPLE 4

### Additional Compounds of Formula I

Additional carboxylic acids, esters, ethers, amides, and N-oxides of compounds of formula I useful in the methods disclosed herein include those disclosed in U.S. Pat. Nos. 7,056,935, for example in columns 13-60; 6,943,187, for example in columns 15-30; 6,509,477, for example in columns 12-22; 6,486,151, for example in columns 15-16; 6,291,510, for example in columns 14-37; 6,218,544, for example in columns 11-12; 6,200,972, for example in columns 12-13; 6,194,440, for example in columns 12-14; 6,191,125, for example in column 6; 6,177,455, for example in columns 6-20; 6,054,452, for example in columns 10 and 15-16; 5,925,666, for example in columns 18-21; 5,859,031, for example in columns 9-11 and 17-24; 5,846,979, for example in columns 7, 10, and 15-17; 5,801,187, for example in columns 7, 8, and 11-12; 5,795,908, for example in columns 9-11 and 17-22; 5,786,378, for example in columns 9-11, 14-18; and 5,614,547, for example in columns 7, 8, and 12-21.

Additional alcohol-substituted carbon-linked compounds useful in the methods disclosed herein include those disclosed in U.S. Pat. No. 5,650,521, for example in columns 6-10.

Additional thioesters and ketones of formula I useful in the methods disclosed herein include those disclosed in U.S. Pat. No. 6,984,639, for example in columns 11-25; U.S. Pat. No. 6,417,209, for example in columns 11-29; U.S. Pat. No. 6,274,607, for example in columns 15-16 and 20-27; U.S. Pat. No. 6,218,424, for example in columns 11-27; U.S. Pat. No. 6,184,243, for example in columns 11, 14-18; U.S. Pat. No. 5,990,131, for example in columns 7-12 and 15-18; U.S. Pat. No. 5,958,949, for example in columns 12-13, 16-20, and 22-30, for example in columns; for example in columns;

Additional heterocyclic compounds of formula I useful in the methods disclosed herein include those disclosed in U.S. Pat. Nos. 6,417,189, for example in columns 30-40; and 6,251,892, for example in columns 15-16.

Additional sulfonyl compounds of formula I useful in the methods disclosed herein include those disclosed in U.S. Pat. Nos. 6,187,806, for example in columns 14-15 and 18-21, and 6,004,993, for example in columns 8 and 14-21.

Each of these applications is hereby incorporated by reference as if written herein in its entirety.

It is expected that some of the foregoing compounds will have activity similar to that described for GM1485, below. All IUPAC names were generated using Cambridge Soft's ChemDraw 10.0.

Stem cells may be organized into a differentiation hierarchy ranging from totipotent stem cells, that are able to form both embryo and placenta; to pluripotent stem cells, that are able only to form the embryo, but have lost the capacity to form the trophoblast (which gives rise to the placenta); to multipotent stem cells, of the three germ layers (endoderm, mesoderm and ectoderm); to monopotent, partially differentiated, tissue-committed populations of stem cells.

The large and growing variety of cells having a differentiation potential similar to or approximating that of embryonic stem cells has made it difficult to precisely define a stem cell, and creates a class of cells that are usefully described as being “stem-like”. As used herein, a “stem-like cell” is a cell capable of giving rise not only to a biological replica of itself, but also to a more differentiated cell. That is, a stem-like cell has the ability to give rise to another stem-like cell that retains the same differentiation potential and may also give rise to a more differentiated cell such as, for example a neuron or hepatocyte.

Oct4 is a mammalian transcription factor, whose expression is exclusively associated with the stem cell phenotype, both in the embryo and in cells that are either derived from the embryo or driven toward a stem cell state by reprogramming. Accordingly, the detection of Oct4 in a cell identifies the cell as having a stem-like phenotype.

It is disclosed herein that adult mammalian fibroblasts reprogram into stem-like cells that express Oct4 when cultured in a medium containing GM1485, in contrast to fibroblasts cultured without GM1485, which fail to reprogram into stem-like cells that express Oct4. Accordingly, provided herein is a use for GM1485 and a method for deriving stem cells from adult mammalian fibroblasts comprising contacting the cells with GM1485.

### Oct4 as a Marker of Differentiated Cells

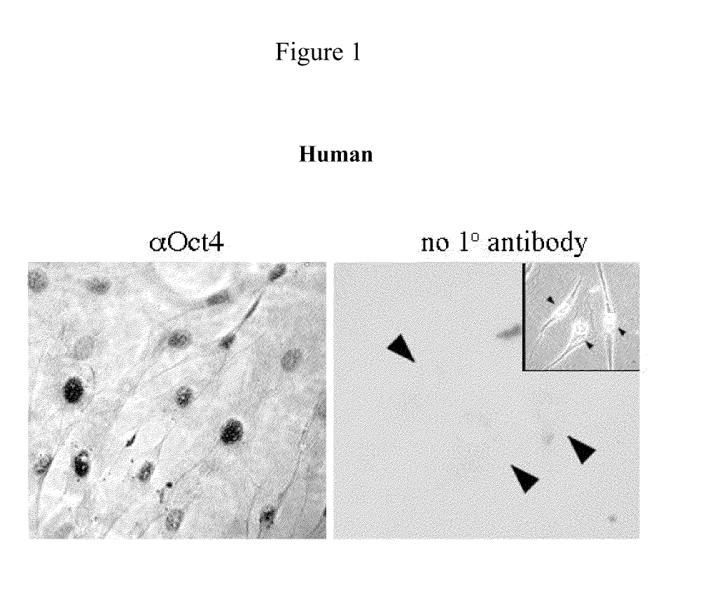

FIG. 1 shows a set of 2 photomicrographs. The upper left photomicrograph shows human fibroblasts treated for three days with GM1485. The upper right photomicrograph shows human fibroblasts treated with a vehicle. The fibroblasts were harvested from a skin sample was taken from the face of a human cadaver. The skin sample was cut into small fragments and digested with dispase overnight (4° C.). Thereafter the epidermis was separated from the dermis. The dermis fragments were further digested with collagenase for approximately 1 h at 37° C., and then diluted with culture medium and filtered to obtain a suspension of dermal cells. The dermal cells were next purified via magnetic bead separation to yield normal human dermal fibroblasts (“NHDFs”). Second passage normal human dermal fibroblasts (NHDF) were seeded onto poly-D-lysine coated coverslips within wells of 24-well plates, at a density of 4,000 cells/well and cultured overnight in NHDF growth medium from PromoCell GmbH(NHDF GM) consisting of:

TABLE 1

| Component | Final Concentration |
|---|---|
| Fibroblast Growth Medium 2 | 1X |
| (PromoCell GmbH) | |
| FCS | 2% |
| Insulin | 5 μg/mL |
| bFGF | 1 ng/mL |

The NHDF GM was aspirated, the wells were rinsed twice with Hank's balanced salt solution (HBSS), and 400 μL of serum free medium (SFM) supplemented with GM1485 at a final concentration of 100 μM or the vehicle the drug vehicle (SFM). The SFM consisted of:

TABLE 2

| | |
|---|---|
| Component | Final Concentration |

| DMEM/F12 50:50 mix supplemented | 1X | |
|---|---|---|
| with L-glutamine and 15 mM HEPES | | |
| Rat Transferrin | 5.5 | ng/mL |
| Sodium Selenite | 5 | μg/mL |
| T₃ | 1X | |
| Putrescine | 1X | |
| Progesterone | 1X | |
| Penicillin-Streptomycin solution | 100 U/mL and 100 mg/mL | |
| Non-Essential Amino Acids (Glycine, | 7.5 μg/mL, 8.9 μg/mL, | |
| L-Alanine, L-Asparagine, L-Aspartic | 13.2 μg/mL, 13.3 μg/mL, | |
| acid, L-Glutamic Acid, L-Proline and | 14.7 μg/mL, 11.5 μg/mL | |
| L-Serine) | and 10.5 μg/mL | |
| Glutamaxx | 2 | mM |
| Fungisome | 625 | mg/mL |
| BSA | 0.5 | mg/mL |
| Glucose | 0.2% | |
| Insulin | 10 | μg/mL |

NHDF cells were cultured accordingly for 72 hours, then fixed with 100% methanol plus 0.3% hydrogen peroxide, at −20° C. for 25 minutes, rinsed with tris-buffered saline (TBS) twice, then blocked at room temperature (RT) for 1 hour with Pierce's TBS Superblock (SB), supplemented with 0.5% triton X-100, and avidin-blocking solution (Vector) was applied for 30 minutes at RT. Coverslips were rinsed in TBS, treated with Vector's biotin blocking solution for 30 minutes at RT and rinsed twice in TBS. The cells were incubated overnight at 4° C. with primary antibody solution, with or without primary antibody:
Mouse monoclonal anti-OCT 3/4
   Santa Cruz Biotechnologies (C-10)
   1:50 in Superblock TBS supplemented with 0.05% triton X-100 (SB Ab buffer)

After removal of primary antibody solutions, the cells were washed three times at RT with TBS and gentle agitation (for 5 minutes each), and incubated for 1 hour at RT with Vector's biotinylated horse anti-mouse IgG at 8 μg/mL (in 2% normal horse serum in TBS supplemented with 0.05% triton X-100). The secondary antibody solutions were removed and cells were washed three times for 5 minutes each at RT, with TBS and gentle agitation. A final incubation was performed with Pierce's neutravidin-HRP at 8 μg/mL in SB Ab buffer, for 45 minutes at RT. After removal of the HRP conjugate, the cells were washed as above with TBS, incubated in VIP (Vector) substrate solution for up to 5 minutes, then washed with ddH₂O for 5 minutes at RT, dehydrated in an ethanol gradient and mounted using Fisher's permount.

The photomicrographs of FIG. 1 were taken using bright field microscopy through a 20× objective, except the inset, which is a phase contrast photomicrograph of the same microscopic field as the bright field photomicrograph into which it is set. The arrows point to the corresponding nuclei in the bright field and phase photomicrographs. The phase contrast photomicrograph demonstrates the presence of cells in the vehicle treated human fibroblasts, even though they are not visible in bright field image because they do not express Oct4.

FIG. 1 demonstrates that treating human fibroblast cells with GM1485 results in their upregulation of Oct4, a POU transcription factor that is a marker of embryonic stem cells, and suggests that these cells may have acquired a stem cell-like phenotype. The differentiation potential of these cells was further tested, as described below.

### GM1485-Mediated Reprogramming of Human Fibroblasts, a Mesodermal-Derived Cell, to a Stem-Cell-Like State, and the Plasticity of GM1485-Mediated Reprogrammed and Further Reprogrammed to Neuroectodermal Cells: Neurons or Astrocytes

On day one of this experiment, the human fibroblasts, were plated, grown overnight as described above and then changed to DMEM supplemented with 10% heat inactivated FCS, 1% DMEM non-essential amino acids (Gibco), penicillin/streptomycin/glutamine (Gemini Bioproducts) and amphotericin B with or without GM1485 for 1 day, and reduced the concentration of serum over an 16 h period until the serum was absent, however the concentration of GM1485 was maintained at 100 μM throughout. The cells were maintained in SFM+/−GM1485 and 10 ng/mL FGF2 for 72 hours.

On day 4 of the experiment, the culture environment was changed to 0.5 mL of a SFM without FGF2, but with rhEGF at 20 ng/ml. The cells were maintained in SFM supplemented with 20 ng/ml rhEGF for an additional 7 days.

On the 11th day all the cells were fixed by aspirating the medium from the wells and replacing it with 0.5 mL/well of 4% paraformaldehyde in phosphate buffered saline (“PBS”) for 15 minutes at room temperature. Thereafter, the cells were briefly rinsed with PBS at room temperature, then washed with PBS at room temperature in three cycles of 5 minutes each. The final PBS wash was removed and the cells were incubated in 10% normal goat serum plus 0.5% of the non-ionic surfactant Triton® X-100 in 1× PBS (blocking/permeabilization buffer) overnight at 4° C. The blocking/permeabilizing buffer was removed and replaced with 0.3 mL/well of a primary antibody wash comprising 2% normal goat serum (“NGS”), 0.05% of the non-ionic surfactant Triton® X-100, 1×PBS, and primary antibodies as follows:

Antibodies for astrocytic markers:
Rabbit α glial fibrillary acidic protein (“GFAP”) (Dako) used at 1:100

Antibodies for neuronal markers:
Mouse Class III β-tubulin (“TuJ-1”) (Covance) used at 1:500

The cells were and incubated in primary antibody for 1 hour at room temperature (“RT”). Thereafter, the primary antibody was removed. The cells were rinsed with PBS at RT, and then washed with PBS at RT in two cycles of 5 minutes each. The cells were then incubated for 1 hour at RT with 0.3 mL/well of a secondary antibody comprising 2% NGS, 0.05% Triton® X-100, 1×PBS, and one of the following fluoro-conjugated detection antibodies:

Goat α rabbit F(ab′)₂ fragment-FITC (Jackson Immunoresearch) used at 25 μg/mL (1:60); or

Goat α mouse F(ab′)₂ fragment-Alexa Fluor 594 (Molecular Probes) used at 8 μg/mL (1:250).

Thereafter the secondary antibody wash was removed. The cells were rinsed with PBS at RT and incubated at RT with Hoechst reagent (Molecular Probes) at 1:1000 in PBS, for 20 seconds. The Hoechst reagent was removed. The cells were then rinsed with PBS at RT, and washed with PBS in two cycles of 5 minutes each, then the cover slips were mounted on a microscope slide in Gel/Mount (Biomeda).

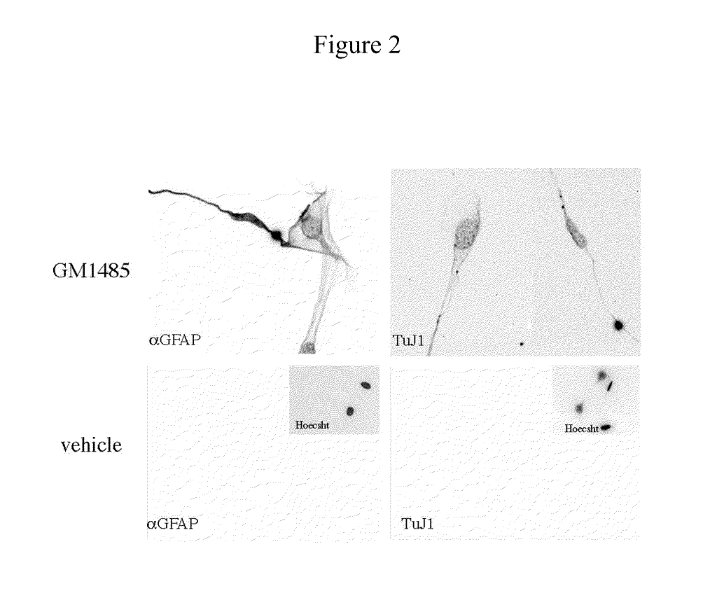

FIG. 2 is comprised of a set of 4 fluorescence photomicrographs, shown in inverted mode to allow full visualization in grayscale. The photomicrographs are arrayed in a matrix of two rows and two columns. The images in the upper row are of cells that had been treated with GM1485 and subsequently cultured under neural inducing conditions, as described above. The photomicrographs in the bottom row had been treated identically with the exception that they were never exposed to GM1485. The cells in the photomicrographs in the left column were stained with an antiserum that recognizes the astrocyte-specific protein glial fibrillary acidic protein (GFAP). The cells in the photomicrographs in the right column were stained with TuJ1, a mouse monoclonal antibody that recognizes neuron-specific βIII tubulin. The photomicrograph insets in the bottom row of photomicrographs are the Hoescht stained nuclei of the cells in the photomicrographs in which they are set. The cells in these photomicrographs were not treated with GM1485, and thus were not reprogrammed along a neural pathway. As a result, these cells express neither GFAP nor neuron-specific βIII tubulin.

In another experiment, the murine fibroblasts, 3T3 cells were cultured in accordance with the protocol described, supra., for Example 1, except that they were not entered into a neural induction environment. Rather, the murine fibroblasts were fixed and stained with rabbit αOct4 antibody.

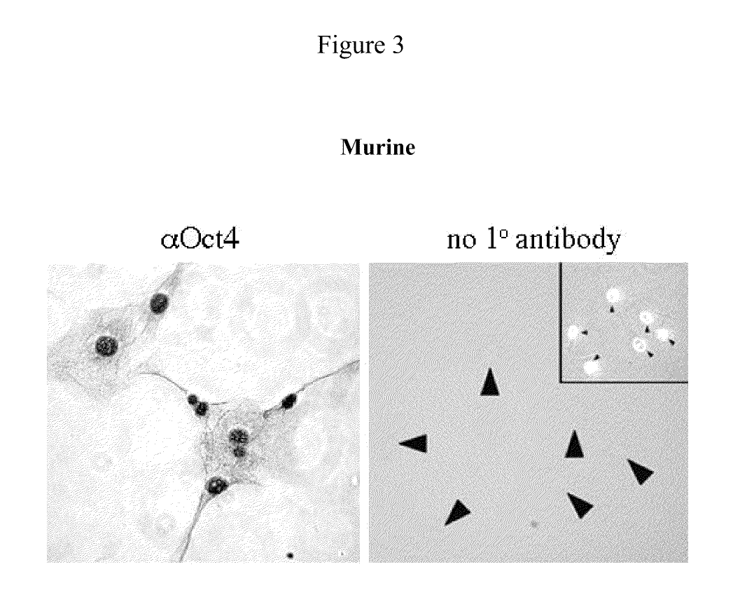

The photomicrographs in FIG. 3 demonstrate that the treatment of rodent fibroblasts with GM1485 results in reprogramming as evidenced by the expression of Oct4 only in the drug treated.

The photomicrographs in FIG. 3 were taken using bright field microscopy using a 20× objective, except the inset, which is a 20× phase contrast photomicrograph of the same microscopic field into which it is set. The arrows point to the corresponding nuclei in the bright field and phase micrographs of the vehicle treated murine fibroblasts. Only the GM1485 treated murine fibroblasts express Oct4.

### Regeneration Through the Reprogramming of and Recruitment of Stem-Like Cells to the Traumatized Tissue.

Under normal conditions, following ischemic damage to the mammalian heart a scar forms and the infarcted tissue does not contribute to electrical conductivity in the heart, nor does it contribute to contractility. Notably, cardiac tissue is believed to be incapable of regeneration and the identification of an adult cardiac stem cell has not been identified. Thus a treatment that is able to induce cardiac regeneration utilizing endogenous stem cells would be of great clinical significance.

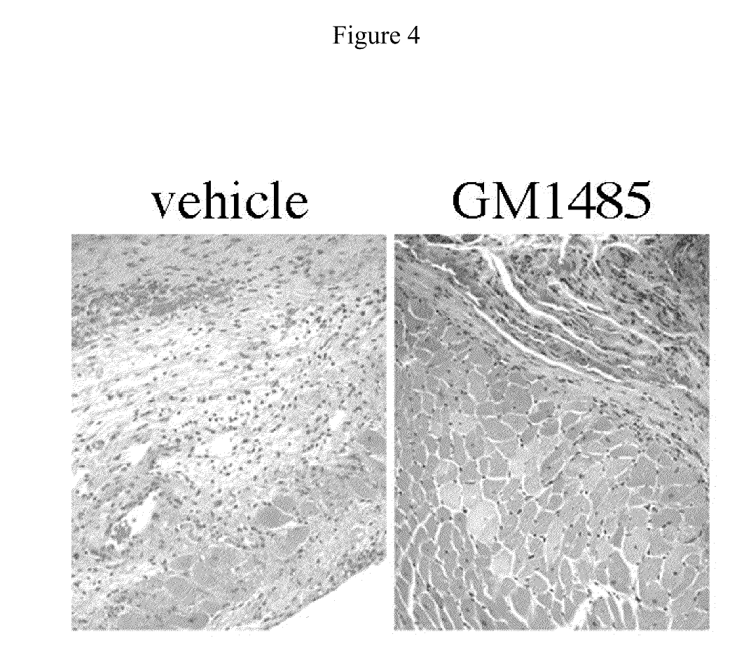

FIG. 4 shows that certain compounds, of which GM1485 is an example, are able to induce Oct4 and Sox2 in vivo, resulting in regeneration following cardiac ischemic injury in adult rats. Adult rats underwent thoracotomy, the heart was isolated, the left anterior descending artery was identified and ligated at its branch point from the left circumflex artery, causing a massive infarction of the left ventricle. The chest was closed, lungs reinflated and the skin closed. The animals began a 30 day regimen of daily intraperitoneal injections of either GM1485 at 5 mg/kg or the saline vehicle in which it was dissolved. On the 30^{th} day after infarction the animals were sacrificed, the hearts harvested, fixed and cryosectioned. The left panel in FIG. 4 shows an H&E stained section of a vehicle treated heart, and the right panel shows an H&E stained section of a GM1485 treated heart. The histology of the vehicle treated tissue is consistent with an extensive myocardial infarction. In contrast, the histology of the GM1485 treated tissue is consistent with normal myocardium.

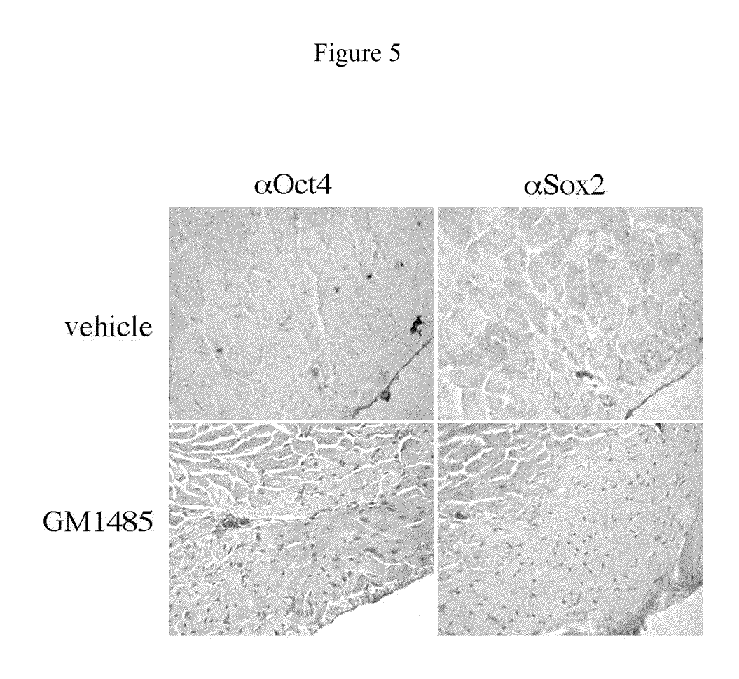

FIG. 5 shows that Oct4 and Sox2 are upregulated in the GM1485-treated, regenerated heart, but not in the vehicle treated tissue. FIG. 5 is a 2×2 matrix of photomicrographs of cardiac tissue 30 days after infarction and treatment with either vehicle in the top two slides or GM1485 in the bottom two slides. The left upper and lower sections were stained with an αOct4 antiserum and the right upper and lower sections were stained with αSox2 (mAb R&D Systems). Only the GM1485 treated cardiac tissue expresses Oct4 and/or Sox2.

In summation, it is herein demonstrated that GM1485 treatment of adult somatic cells can induce the expression of Oct4 and Sox2, and cells that express these proteins acquire the ability to be reprogrammed to an alternative cell fate, (e.g. mesodermal fibroblasts to neuroectodermal neurons or astrocytes), thereby demonstrating that treatment with GM1485 is sufficient to induce a stem cell phenotype. Moreover, these observations are recapitulated in vivo following trauma: the administration of GM1485 participates in the induction of Oct4 and Sox2 expression is critical to the regeneration of a tissue, even a tissue such as the heart that does not regenerate in the absence of Oct4 and Sox2 expression.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method for reprogramming a mammalian somatic cell into a stem-like cell, comprising introducing a small molecule which induces the expression of Oct4.

2. A method for reprogramming a mammalian somatic cell into a stem-like cell, comprising introducing a small molecule which induces the expression of Sox2.

3. The method as recited in either claim 1 or claim 2, wherein the small molecule is an immunophilin ligand.

4. The method as recited in either claim 1 or claim 2, practiced in vitro and additionally comprising the step of culturing the mammalian somatic cell.

5. The method as recited in claim 1, further comprising, after the step of introducing the small molecule which induces the expression of Oct4, culturing the mammalian somatic cell under conditions suitable for maintaining pluripotent stem cells in an undifferentiated state.

6. The method as recited in claim 2, further comprising, after the step of introducing the small molecule which induces the expression of Sox2, culturing the mammalian somatic cell under conditions suitable for maintaining pluripotent stem cells in an undifferentiated state.

7. The method as recited in either claim 5 or claim 6, after the step of introducing the small molecule, culturing the mammalian somatic cell in a serum-free neuro-differentiation medium with FGF2.

8. The method as recited in claim 1, further comprising, after the step of introducing the small molecule which induces the expression of Oct4, culturing the mammalian somatic cell under conditions that induce or direct partial or complete differentiation to a particular cell type.

9. The method as recited in claim 2, further comprising, after the step of introducing a small molecule which induces the expression of Sox2, culturing the mammalian somatic cell under conditions that induce or direct partial or complete differentiation to a particular cell type.

10. The method as recited in either claim 8 or claim 9, further comprising, after the step of culturing the mammalian somatic cell in the neuro-differentiation medium without FGF2, culturing the mammalian somatic cell the neuro-differentiation medium without FGF2 and augmented with rhEGF.

11. The method as recited in claim 1, further comprising, after the step of introducing a small molecule which induces the expression of Oct4, culturing the mammalian somatic cell in a serum-free neuro-differentiation medium.

12. The method as recited in claim 2, further comprising, after the step of introducing a small molecule which induces the expression of Sox2, culturing the mammalian somatic cell in a serum-free neuro-differentiation medium.

13. The method as recited in either claim 11 or claim 12, further comprising, after the step of culturing the mammalian somatic cell in the neuro-differentiation medium without FGF2 and augmented with rhEGF, culturing the mammalian somatic cell the neuro-differentiation medium without FGF2 and augmented with rhEGF and an immunophilin ligand.

14. The method as recited in either claim 1 or claim 2, wherein the mammalian somatic cell is selected from the group consisting of fibroblasts, B cells, T cells, dendritic cells, keratinocytes, adipose cells, epithelial cells, epidermal cells, chondrocytes, neural cells, cardiac cells, esophageal cells, muscle cells, melanocytes, hematopoietic cells, macrophages, monocytes, and mononuclear cells.

15. The method as recited in either claim 1 or claim 2, wherein the mammalian somatic cell is a fibroblast.

16. The method as recited in either claim 1 or claim 2, wherein the mammalian somatic cell is a dermal fibroblast.

17. The method as recited in claim 3, wherein the immunophilin ligand is a compound of the formula XII: 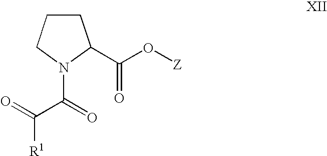
or a pharmaceutically acceptable salt, ester, or hydrate thereof, wherein:
R¹ represents a C₃-C₆ straight or branched chain alkyl or alkenyl group optionally substituted with C₃-C₇ cycloalkyl, C₅-C₇ cycloalkenyl, or phenyl, any of which may be optionally substituted with C₁-C₄ alkyl, C₁-C₄ alkenyl, C₁-C₄ alkoxy, C₁-C₄ haloalkyl, C₁-C₄ haloalkoxy, perfluoromethyl, perfluoromethoxy, halogen, cyano, or hydroxyl; and
Z represents hydrogen.

18. The method as recited in claim 3, wherein the immunophilin ligand is a pharmaceutically acceptable salt or hydrate of a pyrrolidine carboxylic acid.

19. The method as recited in claim 3, wherein the immunophilin ligand is (2S)-1-(1,2-dioxo-3,3-dimethylpentyl)-2-pyrrolidinecarboxylic acid.

20. The method as recited in claim 3, wherein the immunophilin ligand is a pharmaceutically acceptable salt or hydrate of (2S)-1-(1,2-dioxo-3,3-dimethylpentyl)-2-pyrrolidinecarboxylic acid.

21. The method as recited in claim 3, wherein the immunophilin ligand is selected from the group consisting of:
(2S)-1-(3,3-dimethyl-1,2-dioxopentyl)-2-pyrrolidine carboxylic acid,
(2S)-1-(1,2-dioxo-2-cyclohexyl)ethyl-2-pyrrolidinecarboxylic acid,
(2S)-1-(1,2-dioxo-4-cyclohexyl)butyl-2-pyrrolidinecarboxylic acid,
(2S)-1-(3,3-dimethyl-1,2-dioxo-4-hydroxybutyl)-2-pyrrolidine carboxylic acid,
(2S)-1-(3,3-dimethyl-1,2-dioxopentyl)-2-pyrrolidine carboxamide,
1-[1-(3,3-dimethyl-1,2-dioxopentyl)-L-proline]-L-phenylalanine
1-[1-(3,3-dimethyl-1,2-dioxopentyl)-L-proline]-L-leucine,
1-[1-(3,3-dimethyl-1,2-dioxopentyl)-L-proline]-L-phenylglycine,
1-[1-(3,3-dimethyl-1,2-dioxopentyl)-L-proline]-L-phenylalanine, and
1-[1-(3,3-dimethyl-1,2-dioxopentyl)-L-proline]-L-isoleucine.
or a pharmaceutically acceptable salt, ester, or hydrate thereof.

22. The method as recited in either claim 1 or claim 2, wherein the mammal is a human.

23. A method for reprogramming a mammalian somatic cell to become a cell of neural lineage, comprising:
(a) culturing a mammalian somatic cell that is not of neural lineage,
(b) introducing a small molecule which induces the expression of Oct4,
(c) culturing the cell in a serum-free neuro-differentiation medium,
(d) replacing the serum-free neuro-differentiation medium with serum-free neuro-differentiation medium with FGF2,
(e) replacing the serum-free neuro-differentiation medium without FGF2 with serum-free neuro-differentiation medium with FGF2 and augmented with rhEGF,
(f) replacing the serum-free neuro-differentiation medium without FGF2 and augmented with rhEGF with serum-free neuro-differentiation medium without FGF2 and augmented with rhEGF and an immunophilin ligand.

24. The method as recited in claim 23, wherein said small molecule is an immunophilin ligand.

25. The method as recited in claim 23, further comprising assaying to detect a marker of cells of neural lineage.

26. A composition of cells of neural lineage prepared by the method as recited in claim 23.

27. A composition comprising stem-like cells prepared by the method as recited in either claim 1 or claim 2.

28. A method of treatment of a cellular degenerative disorder by the reprogramming of mammalian somatic cell, comprising the administration of a therapeutically effective amount of a small molecule which induces the expression of Oct4 to a patient in need thereof.

29. A method of treatment of a cellular degenerative disorder by the reprogramming of mammalian somatic cell, comprising the administration of a therapeutically effective amount of a small molecule which induces the expression of Sox2 to a patient in need thereof.

30. The method as recited in either claim 28 or claim 29, wherein said small molecule is an immunophilin ligand.

31. A method of treatment, by the reprogramming of mammalian somatic cell, of a disease selected from the group consisting of osteoarthritis, bone fractures, non-union bone fractures, articular trauma, acute coronary syndrome, occlusive stroke, spinal cord injury, traumatic brain injury, peripheral nerve trauma, non-autoimmune demyelinating diseases, acute amyotrophic sclerosis, Huntington's Disease, Alzheimer's Disease, Guillain-Barrè Syndrome, transverse myelitis, hepatic cirrhosis, hepatic fibrosis, macular degeneration, retinal trauma, diabetic retinopathy, actinic keratosis, basal cell carcinoma, keloid scarring, enhanced scar reduction, burns, diabetic ulcers, stasis ulcers, venous ulcers, peptic ulcer disease, duodenal ulcer disease, esophageal lesions, irritable bowel syndrome, periodontal disease, dental implants, acute myelocytic leukemia, acute promylocytic leukemia, breast cancer, cervical cancer, lymphoid cancers and other diseases in which tissue regeneration is a component of healing comprising the administration of a therapeutically effective amount of a small molecule which induces the expression of Oct4 to a patient in need thereof.

32. A method of treatment, by the reprogramming of mammalian somatic cell, of a disease selected from the group consisting of osteoarthritis, bone fractures, non-union bone fractures, articular trauma, acute coronary syndrome, occlusive stroke, spinal cord injury, traumatic brain injury, peripheral nerve trauma, non-autoimmune demyelinating diseases, acute amyotrophic sclerosis, Huntington's Disease, Alzheimer's Disease, Guillain-Barrè Syndrome, transverse myelitis, hepatic cirrhosis, hepatic fibrosis, macular degeneration, retinal trauma, diabetic retinopathy, actinic keratosis, basal cell carcinoma, keloid scarring, enhanced scar reduction, burns, diabetic ulcers, stasis ulcers, venous ulcers, peptic ulcer disease, duodenal ulcer disease, esophageal lesions, irritable bowel syndrome, periodontal disease, dental implants, acute myelocytic leukemia, acute promylocytic leukemia, breast cancer, cervical cancer, lymphoid cancers and other diseases in which tissue regeneration is a component of healing comprising the administration of a therapeutically effective amount of a small molecule which induces the expression of Sox2 to a patient in need thereof.

33. The method as recited in either claim 31 or claim 32, wherein said small molecule is an immunophilin ligand.

34. A method of treatment of a diseases by the reprogramming of mammalian somatic cell, comprising the administration of:
a. a therapeutically effective amount of a small molecule which induces the expression of either Oct4 or Sox2; and
b. another therapeutic agent.

35. The method as recited in claim 34 wherein said other agent is selected from the group consisting of topical or injectable lidocaine, a topical antibiotic, hyaluronan, a long-chain polymer containing repeating disaccharide units of Na-glucuronate-N-acetylglucosamine, with or without chondroitin sulfate, a hydrogel, a nonsteroidal anti-inflammatory drug, collagens or synthetic fillers, topical or oral retinoids, sodium bicarbonate, a pressor agents, Plavix®, a tissue plasminogen activator, streptokinase, and a drug for the treatment of an acute coronary syndromes.

36. A method for achieving an effect in a patient by the reprogramming of mammalian somatic cell, comprising the administration of a therapeutically effective amount of a small molecule which induces the expression of Oct4 to a patient, wherein the effect is selected from the group consisting of enhanced regeneration of donor liver in living-donor liver transplantation, enhanced regeneration of recipient liver in living-donor liver transplantation renal degenerative diseases, enhanced regeneration of skin-graft donor sites, dermal regeneration following surgical or traumatic wounds.

37. A method for achieving an effect in a patient by the reprogramming of mammalian somatic cell, comprising the administration of a therapeutically effective amount of a small molecule which induces the expression of Sox2 to a patient, wherein the effect is selected from the group consisting of enhanced regeneration of donor liver in living-donor liver transplantation, enhanced regeneration of recipient liver in living-donor liver transplantation renal degenerative diseases, enhanced regeneration of skin-graft donor sites, dermal regeneration following surgical or traumatic wounds.

38. The method as recited in either claim 36 or claim 37, wherein said small molecule is an immunophilin ligand.

39. The method as recited in claim 38, wherein said immunophilin ligand is GM1485.
